(19) United States
(12) Patent Application Publication
(10) Pub. No.: US 2013/0345747 A1
(43) Pub. Date: Dec. 26, 2013

(54) BIOLOGICAL SUTURE ANCHOR WITH SUTURE EYELET

(71) Applicant: Arthrex, Inc., Naples, FL (US)

(72) Inventor: Peter J. Dreyfuss, Naples, FL (US)

(73) Assignee: Arthrex, Inc., Naples, FL (US)

(21) Appl. No.: 13/870,314

(22) Filed: Apr. 25, 2013

Related U.S. Application Data
(60) Provisional application No. 61/661,865, filed on Jun. 20, 2012.

(51) Int. Cl. A61B 17/04

(52) U.S. Cl. CPC A61B 17/0401
USPC 606/232

(57) ABSTRACT

A suture anchor for fixation of soft tissue to bone with a body formed of biological material, such as collagen, and a suture eyelet extending through a cannulation of the body. The body may be in the form of a cannulated member or multiple cannulated members. The suture anchor is placed into a pilot hole and, by combination of shape distortion, friction or insertion force, is fixed in place. Sutures are used to re-approximate tissue to bone.

This application claims the benefit of U.S. Provisional Application No. 61/661,865 filed Jun. 20, 2012, the disclosure of which is incorporated by reference in its entirety herein.

### FIELD OF THE INVENTION

The present invention relates to surgical devices and, in particular, to suture anchors and methods of repairing or fixation of soft tissue to bone.

### BACKGROUND OF THE INVENTION

When soft tissue such as a ligament or a tendon becomes detached from a bone, surgery is usually required to reattach or reconstruct the tissue. Often, a tissue graft is attached to the bone to facilitate regrowth and permanent attachment. Techniques and devices that have been developed generally involve tying the soft tissue with suture to an anchor fixed in a hole provided in the bone.

It would be desirable to provide a biological suture anchor comprising at least one collagen member and a separate suture eyelet that allows one or more arthroscopic sliding sutures to be passed therethrough yet providing resistance to cut-through during sliding knot techniques and improved fixation relative to the at least one collagen member.

### SUMMARY OF THE INVENTION

The present invention provides a suture anchor with a body formed of biological material, such as collagen, for fixation of soft tissue to bone. The suture anchor also comprises a suture eyelet extending through a cannulation of the fixation device. The suture anchor is placed into a pilot hole and, by combination of shape distortion, friction or insertion force, is fixed in place. Sutures are used to re-approximate tissue to bone.

The present invention also provides methods of forming a forming a suture anchor with a body formed of biological material, such as collagen, by the steps of: (i) providing a loop with a knot formed by tying two free ends of a flexible strand; and (ii) threading the loop through at least one cannulated member formed of biological material, so that the member stops on the knot at one end of the member and forms an eyelet at the other end of the member.

These and other features and advantages of the invention will be more apparent from the following detailed description that is provided in connection with the accompanying drawings and illustrated exemplary embodiments of the invention.

### BRIEF DESCRIPTION OF THE DRAWING

FIG. 1 illustrates steps of assembling a collagen suture anchor according to a first exemplary embodiment of the present invention.

FIG. 2 illustrates steps of assembling a collagen suture anchor according to a second exemplary embodiment of the present invention.

FIG. 3 illustrates the collagen suture anchor of FIG. 1 with tying sutures attached to the eyelet and in the vicinity of a bone tunnel or socket.

FIG. 4 illustrates the collagen suture anchor of FIG. 3 inserted into the bone tunnel or socket.

### DETAILED DESCRIPTION OF THE EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The present invention provides fixation devices such as suture anchors with an anchor body made substantially of suture and a biological material. The anchor body is formed of one or multiple cannulated members that are formed of biological material such as collagen. The anchor body may preferably comprise biological material throughout its whole length. The biological material (for example, the collagen) may be also provided throughout the length of the suture passing through the anchor body, as desired.

The present invention also provides surgical systems and methods for soft tissue (ligament, tendon, graft, etc.) repair and fixation, such as fixation of soft tissue to bone. In an exemplary embodiment, and as detailed below, the fixation device of the present invention is a collagen-based suture anchor provided with a separate suture eyelet component and a collagen body adjacent the eyelet component. The separate eyelet component provides resistance to cut-through during sliding knot techniques and improved tissue fixation relative to the collagen body. The collagen body consists of one or more collagen cannulated members having various geometries, for example, cylindrical, oval, or round among others.

According to an exemplary embodiment, the present invention provides a collagen suture anchor for fixation of soft tissue to bone which is composed of a suture eyelet or loop, sutures for tying attached to the eyelet, and a collagen cannulated member. The collagen anchor is placed in a pilot hole and, by combination of shape distortion, friction or insertion force, is fixed in place. Sutures are used to re-approximate tissue to bone.

The present invention also provides methods of forming/assembling a fixation device (suture anchor) with a body formed essentially of biological material and suture, by the steps of: (i) providing a suture loop with a knot formed by tying two free ends of a suture strand; and (ii) threading the suture loop through at least one cannulated member formed of biological material, so that the member stops on the knot at one end of the member, and forms a suture eyelet at the other end of the member. Preferably, the at least one cannulated member consists essentially of biological material such as collagen.

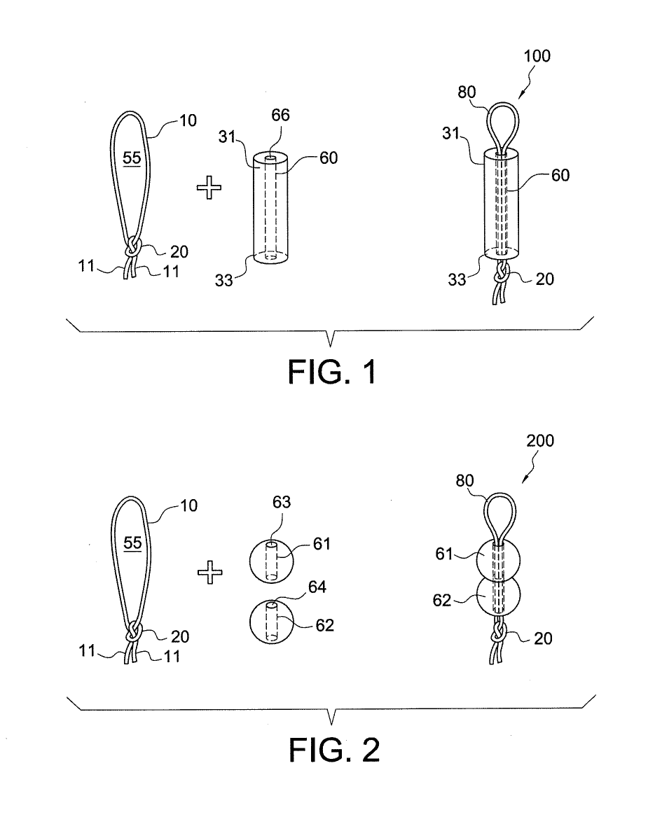

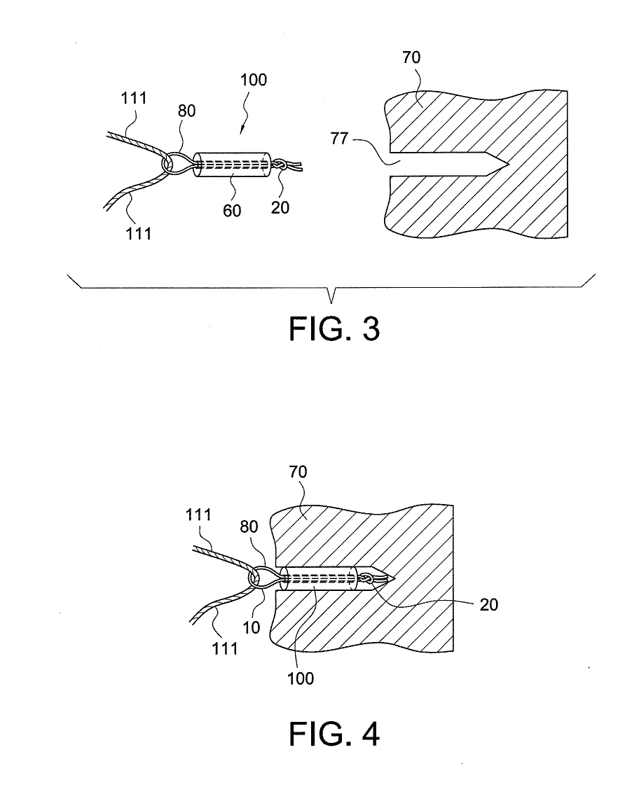

Referring now to the drawings, where like elements are designated by like reference numerals, FIGS. 1 and 2 illustrate exemplary fixation devices 100, 200 (collagen suture anchors 100, 200) of the present invention. FIGS. 3 and 4 illustrate a method of securing a first tissue to a second tissue (for example, soft tissue to bone) with the exemplary fixation devices 100, 200 of the present invention.

FIG. 1 illustrates exemplary fixation device 100 with a body formed of at least one cannulated member 60 consisting essentially of biological material such as collagen, and at least a flexible closed loop 55 (eyelet 55) of a flexible strand 10 extending throughout the cannulated member 60. Flexible strand 10 (for example, suture such as FiberWire® suture or TigerWire® suture 10) forms closed loop 55 when the two free ends 11 of suture 10 are brought together in knot 20. Flexible strand 10 may be formed of suture or similar materials. Closed loop 55 is provided throughout the length of the body of cannulated member 60 and exits at a most proximal end 31 of the cannulated member 60 forming eyelet (loop) 80. Static knot 20 is provided at a most distal end 33 of the cannulated member 60.

Cannulated member 60 may preferably comprise biological material throughout its whole length. The biological material (for example, collagen) may be also provided throughout the entire length or throughout only portions of the anchor body, as desired. Cannulated member 60 may have a cylindrical configuration and/or a diameter that is constant throughout the whole length of cannulated member 60, or may vary depending on the nature of the surgical application and of the instruments with which the suture anchor is employed.

Cannulated member 60 may have the configuration of a cylinder, a sphere, a cone, a prism (for example, triangular or rectangular prism), a cube, a pyramid or similar structures, or combinations of such three-dimensional structures. If desired, the outer surface of cannulated member 60 may be provided with protuberances, indentations or striations (or similar structures such as bulges, bumps and/or prominences) to aid in the fixation of the member within a bone tunnel/socket/hole.

Biological material of cannulated member 60 may include a biopolymer (for example, a bioabsorbable polymer such as collagen or a collagen-based material, among others), or an extracellular matrix protein (such as fibronectin, elastin or laminin, among others). If collagen is employed, the collagen forming cannulated member 60 may be cross-linked, with additional materials, to increase the tensile strength of the collagen strands. The collagen, in its natural state or cross-linked, may be provided in the form of a plurality of collagen strips that are bundled together to form the cannulated member 60 of fixation device 100. The collagen strips may be braided or weaved, for example, or may be provided as parallel strips adjacent to each other, to form the cannulated member 60.

Alternatively, biological material of cannulated member 60 is a spongy biological material which may form a scaffold or matrix containing interstitial spaces and channels that allow cellular invasion and growth (for example, fibroblast proliferation to allow tissue regeneration). The scaffold or matrix may optionally comprise additional components such as blood, proteins, growth factors or chemicals that may be provided (by injection or impregnation, for example) within the matrix.

As such, and in accordance with additional embodiments, cannulated member 60 may be employed to deliver to the surgical site (bone tunnel or socket) a biological component which includes at least one of blood, blood components or fractions, PRP, bone marrow aspirate (BMA) or autologous conditioned plasma (ACP). The biological component may be provided (by injection, impregnation or soaking, for example) directly into the cannulated member 60, and subsequently inserted into the site or into the anatomical tissue. In yet another embodiment, the cannulated member 60 is first inserted into the repair site and then injected with the biological component (for example, PRP, BMA or ACP). If desired, cannulated member 60 may be fabricated to additionally comprise components such as growth factors, additional antiseptic chemicals and/or antibiotics and/or electrolytes, or hormones or site-specific hybrid proteins (that promote or enhance the wound healing effectiveness of the growth factors), or glue such as fibrin glue and/or adhesives, among others.

If retention of the biological components by the cannulated member 60 is desired, at least a portion of the outer surface of the member 60 may be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings.

Cannulated member 60 may have cross-sections of various forms and geometries, including cylindrical round, oval, or rectangular, among others, or combination of such forms and geometries. In an exemplary embodiment only, cannulated member 60 is formed only of biological material, for example, only of collagen. Cannulation 66 may have a diameter about equal to that of a doubled flexible strand 10 (preferably, about few mm greater) to allow closed loop 55 to pass therethrough and yet to stop static knot 20 at distal end 33.

During assembling, closed loop 55 is passed through cannulation 66 of cannulated member 60, to form a separate suture eyelet 80, as shown in FIG. 1. In an exemplary embodiment only, cannulated member 60 is a cylindrical member consisting essentially of collagen and the flexible strand 10 is formed of FiberWire® suture (disclosed in U.S. Pat. No. 6,716,234, the disclosure of which is incorporated herein by reference in its entirety). If desired, the flexible strand 10 is provided with optional colored strands to assist surgeons in distinguishing between suture lengths.

FIG. 2 illustrates another exemplary embodiment of fixation device 200 of the present invention. Fixation device 200 is substantially similar to fixation device 100 of FIG. 1 but differs from it in that its body is formed of a plurality of cannulated members in lieu of the single cannulated member 60, for example, two cannulated round members 61, 62, as also shown in FIG. 2. The plurality of cannulated members are provided as individual body segments attached to each other by the flexible strand 10, and also provided serially along a length of the flexible strand 10, as shown in FIG. 2.

In the specific embodiment shown in FIG. 2, the closed loop 55 is passed through cannulations 63, 64 of the cannulated members 61, 62 to form anchor 200 with separate suture eyelet 80. In embodiments employing more than two cannulated members, the closed loop 55 is passed through at least some of the cannulations of the members, or through all the cannulations of the members.

In an exemplary embodiment only, cannulated members 61, 62 are round or oval members formed of biological material such as collagen. However, and as detailed above with reference to fixation device 100, at least one of cannulated members 61, 62 may comprise any biological material which may include a biopolymer (for example, a bioabsorbable polymer such as collagen or a collagen-based material, among others), or an extracellular matrix protein (such as fibronectin, elastin or laminin, among others). If collagen is employed, the collagen forming cannulated member 61, 62 may be cross-linked, with additional materials, to increase the tensile strength of the collagen strands and of the anchor. Like in the above-described embodiment, the collagen may be provided as a scaffold or matrix that may optionally comprise additional components such as blood, proteins, growth factors or chemicals that may be provided (by injection or impregnation, for example) within the matrix.

At least one of the cannulated members 61, 62 may be an element selected from the group consisting of a cylinder, a sphere, a cone, a prism (for example, triangular or rectangular prism), a cube, a pyramid or similar structures, or combinations of such three-dimensional structures. If desired, the outer surface of at least one of the cannulated members 61, 62 may be provided with protuberances, indentations or striations (or similar structures such as bulges, bumps and/or prominences) to aid in the fixation of the member(s) within a bone tunnel/socket/hole.

The fixation device 100, 200 (collagen suture anchor 100, 200) of the present invention may be employed for attachment of tissue (for example, soft tissue) to bone or other tissue. An exemplary method of tissue repair or fixation with the collagen suture anchor 100, 200 of the present invention comprises the steps of: (i) providing a suture loop 55 of a suture strand 10 by bringing together the two free ends 11 of the strand 10 in a static knot 20; (ii) passing the suture loop 55 through a cannulation 63, 64, 66 of a collagen member 60, 61, 62 to form a collagen suture with a separate suture eyelet 80 and at least one collagen member 60, 61, 62; (iii) attaching at least one tying suture 111 (optionally attached to soft tissue) to the separate suture eyelet 80 of the collagen anchor 100, 200; and (iv) placing the collagen anchor 100, 200 (optionally attached to soft tissue) in a pilot hole 77 formed in bone 70. The suture anchor 100, 200 is fixed in place in the hole by a combination of shape distortion, friction or insertion forces. The sutures are used to re-approximate tissue to bone.

FIGS. 3 and 4 illustrate insertion and fixation of exemplary fixation device 100 of the present invention into bone socket/tunnel/hole 77 formed within bone 70. At least one tying suture 111 is attached to tissue (for example, soft tissue to be repaired). Additional arthroscopic sutures (such as tying or sliding sutures) attached to tissue (such as soft tissue) may be threaded through the eyelet 80 of the collagen suture anchor 100, 200 to allow secure fixation of the tissue to bone, as desired and depending on the specific characteristics of each surgical repair. The separate eyelet component 80 provides resistance to cut-through during sliding knot techniques and improved tissue fixation relative to the collagen body.

Preferably, the suture strand 10 of the collagen suture anchor 100, 200 is formed of suture, for example, FiberWire® suture (disclosed in U.S. Pat. No. 6,716,234) or TigerWire® suture, or suture chain (such as FiberChain® disclosed in U.S. Pat. No. 7,803,173) or suture tape (such as FiberTape® disclosed in U.S. Pat. No. 7,892,256), or nitinol, among others, or combination of these materials.

The collagen suture anchor of the present invention has applicability to repair applications that may be employed in surgical procedures such as rotator cuff repair, Achilles tendon repair, patellar tendon repair, ACL/PCL reconstruction, hip and shoulder reconstruction procedures, and any application used with fixation devices including at least one suture anchor.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting.

What is claimed is:

1. A fixation device, comprising:
an anchor body consisting essentially of biological material; and
at least one flexible strand extending through the anchor body.

2. The fixation device of claim 1, wherein the biological material is collagen.

3. The fixation device of claim 1, wherein the flexible strand forms an eyelet at one end of the anchor body and a static knot at the other end of the anchor body.

4. The fixation device of claim 3, further comprising at least another flexible strand received through the eyelet for further securement to tissue.

5. The fixation device of claim 1, wherein the flexible strand is a suture, a suture tape or a suture chain.

6. The fixation device of claim 1, wherein the flexible strand is a suture formed of ultrahigh molecular weight polyethylene.

7. The fixation device of claim 1, wherein the anchor body is a cannulated cylinder formed only of collagen.

8. The fixation device of claim 1, wherein the anchor body comprises a plurality of individual cannulated members, at least one of the individual cannulated members being formed of collagen.

9. A suture anchor for surgical repairs consisting essentially of a cannulated member formed of collagen and a flexible strand extending through the cannulated member.

10. The suture anchor of claim 9, wherein the cannulated member consists essentially of collagen and the flexible strand is a high strength suture.

11. The suture anchor of claim 9, wherein the flexible strand forms a closed eyelet and a knot, wherein the closed eyelet extends through a cannulation of the cannulated member and exits one end of the cannulated member, and wherein the knot is located at the other end of the cannulated member.

12. The suture anchor of claim 9, wherein the cannulated member is formed of at least one geometrical structure selected from the group consisting of a cylinder, a sphere, a cone, or a prism.

13. The suture anchor of claim 9, wherein the flexible strand is a suture, a suture tape or a suture chain.

14. The suture anchor of claim 9, wherein the cannulated member further comprises a biological component selected from the group consisting of platelet-rich plasma, autologous conditioned plasma and bone marrow aspirate.

15. A suture anchor comprising a plurality of body segments connected by a material strand, at least one of the plurality of body segments being formed essentially of collagen.

16. The suture anchor of claim 15, wherein all of the plurality of body segments are formed essentially of collagen.

17. The suture anchor of claim 15, wherein the plurality of body segments are provided serially along a length of the material strand.

18. The suture anchor of claim 15, wherein the material strand forms an eyelet at a most proximal end of the suture anchor and a knot at a most distal end of the suture anchor.

19. The suture anchor of claim 15, wherein the material strand is one of a suture strand, a nitinol strand, a suture tape or a collagen tape.

20. A method of fixation of a first tissue to a second tissue, comprising the steps of:
providing a suture anchor comprising a length of a flexible strand and at least one cannulated body member formed at least partially around and at least partially along the length of the flexible strand, the flexible strand forming an eyelet at a most proximal end of the suture anchor and a knot at a most distal end of the suture anchor, the cannulated body member consisting essentially of collagen;
threading suture through the eyelet; and
placing the suture anchor into bone.

21. The method of claim 20, wherein the suture anchor secures soft tissue to bone.

22. The method of claim 20, wherein the suture anchor is installed into a bone tunnel or socket.

23. The method of claim 20, wherein the flexible strand is one of a suture strand, a suture tape, a suture chain or a nitinol strand.
